(19) United States
(12) Patent Application Publication
(10) Pub. No.: US 2004/0258632 A1
(43) Pub. Date: Dec. 23, 2004

(54) Stable aqueous antiplaque oral compositions

(76) Inventors: Thomas J. Boyd, Somerset, NJ (US); Guofeng Xu, Princeton, NJ (US); Abdul Gaffar, Princeton, NJ (US); David B. Viscio, Monmouth Jct., NJ (US)

Correspondence Address: Colgate-Palmolive Company, 909 River Road, P.O. Box 1343, Piscataway, NJ, 08855-1343, US

(21) Appl. No.: 10/601,478

(22) Filed: Jun. 23, 2003

(51) Int. Cl. A61K 7/16; A61K 7/22

(52) U.S. Cl. 424/49

(57) ABSTRACT

A stable aqueous antiplaque oral composition containing (a) an antibacterial ester compound having the formula

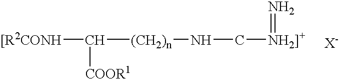

where R¹ is an alkyl chain of 1 to 8 carbon atoms, and R² is an alkyl chain of 6 to 30 carbon atoms and X is an anion, (b) a surfactant; (c) a humectant; and, wherein the composition is free of monohydric alcohol.

### BACKGROUND OF THE INVENTION

1. Field of the Invention

The present invention relates generally to aqueous oral compositions effective in retarding bacterial plaque accumulation on teeth and more particularly to stable aqueous compositions containing an antimicrobial arginine derivative compound.

2. The Prior Art

Dental plaque is a soft deposit which forms on teeth and is comprised of an accumulation of bacteria and bacterial by-products. Plaque adheres tenaciously at the points of irregularity or discontinuity, e.g., on rough calculus surfaces, at the gum line and the like. Besides being unsightly, plaque is implicated in the occurrence of gingivitis and other forms of periodontal disease.

A wide variety of antibacterial agents have been suggested in the art to retard plaque formation and the oral infections associated with plaque formation. For example, U.S. Pat. No. 5,874,068 discloses aqueous oral compositions containing the arginine derivative compound, Nα-acyl amino acid ester and salts thereof, as being effective to counter plaque formation by bacterial accumulation in the oral cavity. According to U.S. Pat. No. 5,874,068, as the N^{α}-lauryl-L-arginine alkyl ester is unstable in aqueous environments such as mouthrinses and generally undergoes hydrolysis reactions typical of esters, the arginine derivative compound being stabilized against hydrolysis by the presence in the mouthrinse of a monohydroxy alcohol represented by the formula ROH where R is an alkyl group containing 1 to 8 carbons, such formula including a monohydroxy alcohol such as ethanol which is present in the mouthrinse at a concentration in the range of about 10 to 35% v/v.

A drawback to the high (10-35% v/v) concentration of alcohol such as ethanol in the aqueous oral compositions as disclosed in U.S. Pat. No. 5,874,068, is that there is a public health concern involving the risk that alcoholic persons may intentionally ingest high alcohol mouthrinses and that children may incur serious injuries due to poisoning from high alcohol mouthrinses and that adolescents may abuse such mouthrinses whereby liquor laws otherwise render alcohol unobtainable.

Other prior art concerned with the antibacterial efficacy of arginine derivative compounds include UK 1352420 which discloses that arginine alkyl ester compounds exhibit antibacterial activity in the oral cavity against bacterium belonging to the genus, Lactobacillus, a main pathogen of dental caries and a bacterium belonging to the genus strapylococcus, a main pathogen of alveolar pyorrhea.

U.S. Pat. No. 5,266,306 discloses an oral composition containing a bactericidal amount of cetylpyridinium chloride and the arginine derivative compound N^{α}-acyl amino acid alkyl ester such as N^{α}-cocoyl-L-arginine methyl ester hydrochloride salt, the arginine derivative compound salt being effective to promote the absorption of cetylpyridinium chloride on tooth surfaces.

Thus there is a clear need in the art to formulate a stable alcohol-free aqueous oral composition such as a mouthrinse capable of delivering an antibacterial arginine derivative compound in amounts effective to retard of bacterial plaque accumulation on teeth without inhibiting the bioavailability of the antibacterial arginine derivative compound.

### SUMMARY OF THE INVENTION

In accordance with the present invention there is provided a chemically stable aqueous oral composition containing an amount of an arginine derivative ester compound and salts thereof, effective to counter plaque formation by bacterial accumulation in the oral cavity, which oral composition is free of a monohydric alcohol which composition is comprised of an aqueous vehicle containing a humectant, a surfactant, and an arginine derivative ester represented by the formula

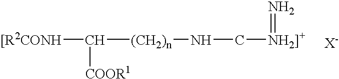

wherein R¹ is an alkyl group having 1 to 8 carbon atoms R² is an alkyl group having 6 to 30 atoms, n is an integer from 1 to 6, X⁻ is an anion.

### DESCRIPTION OF THE PREFERRED EMBODIMENTS

Aqueous Vehicle

Water can comprise from about 50% to about 80% by weight, preferably from about 55% to about 75% by weight of the aqueous oral compositions of the present invention. These amounts of water include the free water which is added, plus that amount which is introduced with other materials.

The aqueous oral composition of the present invention such as a mouthrinse is prepared using a vehicle which contains water and a humectant. The humectant is generally a mixture of humectants, such as glycerin and sorbitol, and a polyhydric alcohol such as propylene glycol, butylene glycol, hexylene glycol, polyethylene glocol. The humectant content is in the range of about 5 to abut 40% by weight and preferably about 10 to about 30% by weight. The water content is in the range of about 50 to about 80% by weight and preferably 55 to about 75% by weight.

Antibacterial Ester

In the above identified antibacterial ester formula, R²CO may be a natural system mixed fatty acid residue such as coconut oil fatty acid, tallow fatty acid residue and the like, or a mono-fatty acid residue such as lauroyl, myristyl, stearoyl and the like, the lauroyl group being preferred.

Examples of antibacterial ester salts of the above identified formula include inorganic acid salts such as hydrochloride, sulfate or an organic salt such as acetate, tautarate or citrate, the chloride salt being preferred.

Examples of antibacterial ester compounds preferred in the practice of the present invention are antibacterial ester compound of the above-identified formula wherein n in the formula equals 3 useful in the practice of the present invention include N^{α}-cocoyl-L-arginine methyl ester, N^{α}-cocoyl-L-arginine ethyl ester, N^{α}-cocoyl-L-arginine propyl ester, N^{α} stearoyl-L-arginine methyl ester, N^{α} stearoyl-L-arginine ethyl ester hydrochloride. The term “cocoyl” is an abbreviation for coconut oil fatty acid residue, and chloride salts of these compounds, these ester compounds and the salts thereof being referred to in this specification as arginine derivative compounds. Arginine derivative compounds and their salts in particular show excellent inhibitory effect against microorganisms which possess relatively strong resistance to bacterial such as S. aureus, S. mutans, F. nucleatum which are involved in plaque formation on teeth. An arginine derivative compound preferred in the practice of the invention is the hydrogen chloride salt of ethyl lauroyl arginine.

The antibacterial ester of the present invention is present in the aqueous oral compositions at a concentration of about 0.05 to about 2.0% by weight and preferably about 0.075 to about 1% by weight.

Surfactant

Surfactants useful in the practice of the present invention include nonionic and zwitterionic surfactants. Suitable nonionic surfactants useful in the present invention include poly(oxyethylene)-poly(oxypropylene) block copolymers. Such copolymers are known commercially by the non-proprietary name of poloxamers, which name is used in conjunction with a numeric suffix to designate the individual identification of each copolymer. Poloxamers may have varying contents of ethylene oxide and propylene oxide which results in poloxamers which have a wide range of chemical structures and molecular weights.

A preferred group of nonionic surfactants useful in the present invention include condensates of sorbitan esters of fatty acids with ethylene oxide (polysorbates) such as sorbitan mono-oleate with from about 20 to about 60 moles of ethylene oxide (e.g., “Tweens”, a trademark of ICI US, Inc.). Particularly preferred polysorbates are Polysorbate 20 (polyoxyethylene 20 sorbitan monolaurate, Tween 20) and Polysorbate 80 (polyoxyethylene 20 sorbitan mono-oleate, Tween 80).

Zwitterion surfactants useful in the practice of the present invention particularly betaine surfactants, include surfactants disclosed in U.S. Pat. No. 5,180,577, incorporated herein by reference. Typical alkyldimethyl betaines include decyl betaine 2-(N-decyl-N,N-dimethylammonio) acetate, cocobetaine or 2-(N-coc-N, N-dimethyl ammonio) acetate, myristyl betaine, palmityl betaine, lauryl, betaine, cetyl betaine, cetyl betaine, stearyl betaine, etc. The amidobetaines are exemplified by cocoamidoethyl betaine, cocoamidopropyl betaine, laurmidopropyl betaine and the like. The preferred betaine is the cocoamidopropyl betaine.

The surfactant is present in the aqueous oral compositions of the present invention range from about 0.1% to about 5% by weight preferably from about 0.6% to about 2.0% by weight.

Other Ingredients

Any suitable flavoring or sweetening material may also be incorporated in the mouthrinse composition of the present invention. Examples of suitable flavoring constituents are flavoring oils, e.g., oils of spearmint, peppermint, wintergreen, sassafras, clove, sage, eucalyptus, marjoram, cinnamon, lemon and orange and methyl salicylate. Suitable sweetening agents include sucrose, lactose, maltose, sorbitol, xylitol, sodium cyclamate, perillartine and sodium saccharin. Suitably, flavor and sweetening agents may together comprise from 0.01% to 5% by weight or more of the mouthrinse composition and at such concentrations render the mouthrinse with a palatability acceptable to the user.

The oral composition of the present invention may additionally contain a fluoridating agent to aid in preventing dental caries as well as antibacterial metal salts, halogenated diphenyl ethers and enzymes. Fluoridating agents suitable for use in the oral compositions of the present invention includes sodium fluoride, potassium fluoride, stannous fluoride and complex fluorides such as sodium monofluorophosphate. The fluoridating agent is most desirably present in an amount to provide 1000-2000 ppm fluoride ion in the composition.

Examples of antibacterial metal salts suitable for use in the present invention include stannous salts such as stannous chloride, stannous gluconate, zinc salts such as zinc chloride, zinc gluconate, zinc citrate and copper salts such as copper gluconate. Examples of halogenated diphenyl ethers include Triclosan and enzymes include papain and glycoamylase. These agents may be present in the composition of the present invention at concentrations of about 0.1 to about 2% by weight.

Antitartar agents compatible with antibacterial esters such as ethyl lauroyl arginine may also be included in the oral composition of the present invention. An example of such antitartar agents include cationic polyphonates such as water soluble quaternary aminoalkylene phosphonic compounds as disclosed in U.S. Pat. No. 4,118,472, the disclosure of which is herein incorporated by reference. These antitartar agents may be included in the oral composition of the present invention at a concentration of about 0.1 to about 5% by weight.

Antitartar agents which are not compatible with antibacterial esters such as pyrophosphate and polyphosphate salts may be included in one component of a dual component oral composition system in which a first component contains the antibacterial ester and the second component contains the incompatible antitartar salt, the first and second components being maintained separate from each other until dispersed and combined for application to the teeth.

The following example further describes and demonstrates preferred embodiments within the scope of the present invention. The example is given solely for illustration, and is not to be construed as limitation of this invention as many variations thereof are possible without departing from its spirit and scope.

### EXAMPLE I

A mouthrinse of the present invention having a pH of 5.0 was prepared by dissolving in water each of the ingredients listed in Table I below with agitation in a glass mixing vessel.

TABLE I

| Ingredient | Wt. % |
|---|---|
| Ethyl lauroyl arginate HCl (ELAH) | 0.1 |
| Sorbitol | 10.0 |
| Glycerin | 10.0 |
| Propylene glycol | 7.0 |
| Polysorbate 20 | 0.8 |
| Cocoamidopropyl betaine | 0.8 |
| Sodium saccharin | 0.03 |
| Flavor | 0.10 |
| Water | Q.S. |

After 9 months at room temperature, the ELAH concentration was determined by Gas Chromatography—Mass Spectrometry to be unchanged at 0.1% by weight.

A double blind randomized clinical study was conducted in which 15 human subjects were asked to rinse for one minute with either the mouthrinse in Example I or a matching placebo (i.e., without ELAH) twice a day for 4 days while forgoing all other maintenance oral hygiene. There was a statistically significant reduction of 11.6% in plaque using the mouth rinse of Table I. The results of the study are recorded in Table II below.

TABLE II
Clinical efficacy of an alcohol-free mouthrinse

| Mouthrinse | Mean QHI* (SD)** | % Reduction rel placebo |
|---|---|---|
| Placebo | 2.51 (0.30) | — |
| 0.1% ELAH | 2.22 (0.22) | 11.6** |

*QHI = Quitley & Hein Index (Art recognized measure of plaque on teeth).
**Standard Deviation
**Significant at the 95% confidence level.

What is claimed is:

1. A stable aqueous antiplaque oral composition comprised of
(a) a safe and effective amount of an antibacterial ester represented by the formula 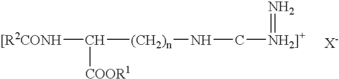
where R¹ is an alkyl chain of 1 to 8 carbon atoms, and R² is an alkyl chain of 6 to 30 carbon atoms and X is an anion,
(b) a surfactant;
(c) a humectant; and,
(d) water
wherein the composition is free of a monohydric alcohol.

2. The composition of claim 1 wherein n=3.

3. The composition of claim 1 wherein the antibacterial ester is the hydrochloride salt of ethyl lauroyl arginine.

4. The composition according to claim 1 wherein the concentration of the antibacterial ester compound is present at a concentration of about 0.02% to 2% by weight.

5. The composition according to claim 1 wherein the surfactant is a mixture of nonionic and zwitterionic surfactants.

6. A composition of claim 1 wherein said polyhydric alcohol is propylene glycol.

7. The composition according to claim 1 wherein the humectant is a mixture of polyhydric alcohols.
